(19) United States
(12) Patent Application Publication
(10) Pub. No.: US 2015/0031989 A1
(43) Pub. Date: Jan. 29, 2015

(54) INDICATOR GUIDE FOR IMPROVED INSTRUMENT NAVIGATION IN IMAGE-GUIDED MEDICAL PROCEDURES

(71) Applicant: CIVCO MEDICAN INSTRUMENTS CO., INC., Kalona, IA (US)

(72) Inventor: Willet F. Whitmore, III, Longboat Key, FL (US)

(21) Appl. No.: 14/372,804

(22) PCT Filed: Jan. 15, 2013
(86) PCT No.: PCT/US2013/021550

§ 371 Date: Jul. 17, 2014

Related U.S. Application Data
(60) Provisional application No. 61/588,905, filed on Jan. 20, 2012.

(51) Int. Cl. A61B 19/00

(52) U.S. Cl. CPC A61B 19/5244; A61B 19/201; A61B 2019/5251; A61B 2019/562
USPC 600/424

(57) ABSTRACT

An indicator guide and method of use is provided for use with an instrument arranged to be guided to a target site within the body of the patient by an electronic tracking system. A sensor is coupled to at least one of the instrument and indicator guide provides a signal to the tracking system which determines the position and orientation of the instrument with respect to the target site and provides output signals indicative thereof. The indicator guide includes a display responsive to the output signals for providing a visual indication of the path the instrument should take to reach the target site and for providing a perceptible indication of the distance to the target site. The display is located on or immediately adjacent the instrument and within the surgical field, whereupon the user can readily see the display while directly viewing and moving the instrument along that path.

### CROSS-REFERENCE TO RELATED APPLICATIONS

This PCT application claims the benefit under 35 U.S.C. §119(e) of Provisional application Ser. No. 61/588,905 filed on Jan. 20, 2012 entitled INDICATOR GUIDE FOR IMPROVED INSTRUMENT NAVIGATION IN IMAGE-GUIDED MEDICAL PROCEDURES whose entire disclosure is incorporated by reference herein.

### STATEMENT REGARDING FEDERALLY SPONSORED RESEARCH OR DEVELOPMENT

“Not Applicable”

### INCORPORATION-BY-REFERENCE OF MATERIAL SUBMITTED ON A COMPACT DISK

“Not Applicable”

### FIELD OF THE INVENTION

This invention relates generally to instruments for image guide diagnostic and therapeutic procedures within the body of a patient and more particularly to guides for providing the user with intuitive visual directions to facilitate the precise move the instrument to a desired target within the body of the patient.

### BACKGROUND OF THE INVENTION

Image guided diagnostic and therapeutic procedures use intermittent or constant (real-time) imaging to guide an instrument to a target lesion. Typically, images are acquired as 3-D volume digital data, while the operator views the images in multi-planar 2-D. This has proven to be the best way for the human brain to deal with the 3-D constructs required for accurate needle placement into a target lesion within a human body. These “live” (either real-time and or very near term, “current”) image data may be enhanced by fusion with previously acquired volume image data if there is a valid method to reference and accurately register the data sets. Electromagnetic (EM) fields and sensors have become accepted as one method for establishing a valid frame of reference for volume image registration and instrument/needle tracking. For example, a small lesion in the prostate may be recognized on an MRI scan using an endo-rectal coil, but may not be visible on ultrasound of the same prostate using an endo-rectal ultrasound transducer for ultrasound guided needle biopsy. If an EM field generator is placed adjacent to the patient's pelvis and a 6 degree of freedom (DOF) EM sensor affixed to the transducer, fusion of the prior MRI scan volume data to the real-time ultrasound image(s) can be achieved as follows: Software in the ultrasound machine allows input of the MRI volume images in DICOM format. This volume MR data is then registered to the transducer and the real-time ultrasound image(s) via a series anatomic reference points common to both imaging modalities that are entered into the software to achieve anatomic registration (typically obtained from real time 2-D ultrasound images with the electromagnetic sensor that is attached to the imaging transducer in a known position and orientation, or by an implanted or externally placed 3-D reference marker). The combination of the EM field and sensor becomes the 3-D frame of reference common to both the prior MRI volume image data and the current real time ultrasound images because the position of the ultrasound transducer is being tracked in real-time by the EM sensor. Thus, the 2-D ultrasound images may be fused in real time with the fully registered 3-D volume data of the MRI scan. In this way, ultrasound may be used to “see” the otherwise invisible target area because it is visible on the overlying MR images. Most importantly for purposes herein, additional EM sensors may be attached to other instruments such as needles for 3-D image guided navigation within this common frame of reference.

Instrument/needle guidance in this setting is the subject of the present invention. To date, needle guidance using either real-time or “current” images is accomplished by the operator watching the digital images on a monitor while manipulating the needle blindly with his/her hands. Using prior art EM navigation and instrument tracking, there is additional iconography on the monitor that uses the sensor position in the needle to create a virtual instrument. Various on-screen software constructs give indications of needle position and trajectory in relation to the screen image. And, if a target lesion is marked, the software will have screen icons and/or coloration that indicate the corrections required to put the needle on the correct target trajectory and provide a distance to the target. This is, at least potentially, a major improvement over currently established practice standards: namely, completely blind freehand needle positioning with frequent re-imaging to see the effects or, trying to follow the needle by direct visualization using real time imaging. However, several problems have slowed adoption of EM systems guidance. First, there is added cost. This can be addressed by data showing reduced procedure times and better outcomes. Second, there are issues related to the EM field and accuracy which are being resolve with better technology. But finally, the main problem with existing systems, which all use this standard software overlay approach on the imaging monitors and employ sensors in the needles as described above, is that they are not simple or intuitive.

When using EM instrument tracking, what the practitioner sees on the monitor (prior art) are current relationships in flashing multi-planar 2-D, or scrolling 2-D sequences combined with the desired direction of corrective movement in some 2-D rendering and/or indicator of the 3-D movement required. Furthermore, as the operator makes corrective movements, his/her hand moves are essentially blind, trial and error that are corrected only by recognition of changes in previously learned software indicators and a 3-D construct in his/her mind. Correctly moving an instrument/needle in three dimensions based on 2-D images seen on a screen that has no particular orientation to the operative field and while not watching the hands requires both skill and practice. In addition, an operator must learn the specifics of the iconography designed into the software that indicate the required needle re-direction as well as interpret the image(s). The result is a steep learning curve that requires expensive (skilled teachers plus time) education and results in a spectrum of skill even amongst experienced users. These non-intuitive requirements have slowed the adoption and delayed realization of the full benefits of this otherwise highly developed and beneficial technology.

The subject invention addresses those needs by providing a new guidance device and method of guidance that allows all operators to bypass the skill requirements of prior art systems, flattens the learning curve and makes image guided instrument/needle placement completely intuitive.

### SUMMARY OF THE INVENTION

In accordance with one aspect of this invention an indicator guide is provided for guiding the movement of an instrument by a user in an image guided medical procedure on a patient. The instrument is arranged to be introduced and guided in a surgical field to a target site within the body of the patient by an electronic tracking system. At least one of the instrument and the indicator guide comprise a sensor for providing a signal to the tracking system. The tracking system is arranged determine the position and orientation of the instrument with respect to the target site in response to the signal from the sensor and for providing output signals to the indicator guide. The indicator guide comprises a display responsive to the output signals from the tracking system for providing a visual indication of the path to which the instrument should be oriented and directed to reach the target site and for providing a perceptible indication of the distance of the instrument to the target site. The display is located on or immediately adjacent the instrument and within the surgical field, whereupon the user can readily see the display while directly viewing and moving the instrument along that path.

In accordance with another aspect of this invention there is provided a method for guiding the movement of an instrument by a user in an image guided medical procedure on a patient. The instrument is arranged to be introduced and guided to a target site within the body of the patient by an electronic tracking system. The tracking system includes a sensor is arranged determine the position and orientation of the instrument with respect to the target site in response to a signal from the sensor and for providing output signals indicative thereof. The method entails providing an instrument guide comprising a display. The display is responsive to the output signals from the tracking system for providing a visual indication of the path to which the instrument should be oriented and directed to reach the target site and for providing a perceptible indication of the distance of the instrument to the target site. The sensor is coupled to at least one of the instrument and the indicator guide. The display is disposed on or immediately adjacent the instrument to be movable with the instrument, whereupon the user can readily see the display while directly viewing and moving the instrument along that path.

### DESCRIPTION OF THE DRAWING

FIG. 1 is a top plan view of one exemplary embodiment of an indicator guide constructed in accordance with this invention shown with an adaptor mounting the guide onto an instrument and so that it can be coupled to an electronic tracking system for use in an image guided medical procedure on a patient;

FIG. 2 is a side elevation view taken along line 2-2 of FIG. 1;

FIG. 3 is a top view of the exemplary embodiment of the indicator guide shown in FIG. 1, but making use of another means, e.g., adhesive tape, for mounting the guide on the instrument or on the hand of a user or on some other structure within the surgical field;

FIG. 4 is a side elevation view taken along line 4-4 of FIG. 3;

FIG. 5A is a block diagram showing one exemplary use of an indicator guide constructed in accordance with the present invention in an electronic tracking system making use of a pair of position sensors, e.g., EM sensors, with the indicator guide arranged to be releasably secured to the instrument and with one of the sensors coupled to the instrument and the other of the sensors coupled to the indicator guide;

FIG. 5B is a block diagram showing another exemplary use of an indicator guide constructed in accordance with the present invention in an electronic tracking system making use of the pair of position sensors like shown in FIG. 5A, but with the indicator guide arranged to be releasably secured to the hand of the user holding the instrument;

FIG. 5C is a block diagram showing yet another indicator guide constructed in accordance with the present invention in an electronic tracking system making use of the pair of position sensors like shown in FIGS. 5A and 5B, but with the indicator guide arranged to be releasably secured to some other structure, e.g., a surgical drape, within the surgical field and the direct vision field of the user holding the instrument;

FIG. 5D is a block diagram showing still another indicator guide constructed in accordance with the present invention in an electronic tracking system making use of only a single position sensor, i.e., a position sensor coupled to the instrument, with the indicator guide being arranged to be releasably secured to the instrument; and

FIG. 5E is a block diagram showing still another indicator guide constructed in accordance with the present invention in an electronic tracking system making use of only a single position sensor, i.e., a position sensor coupled to the indicator guide, with the indicator guide being arranged to be releasably secured to the instrument.

### DETAILED DESCRIPTION OF THE PREFERRED EMBODIMENT

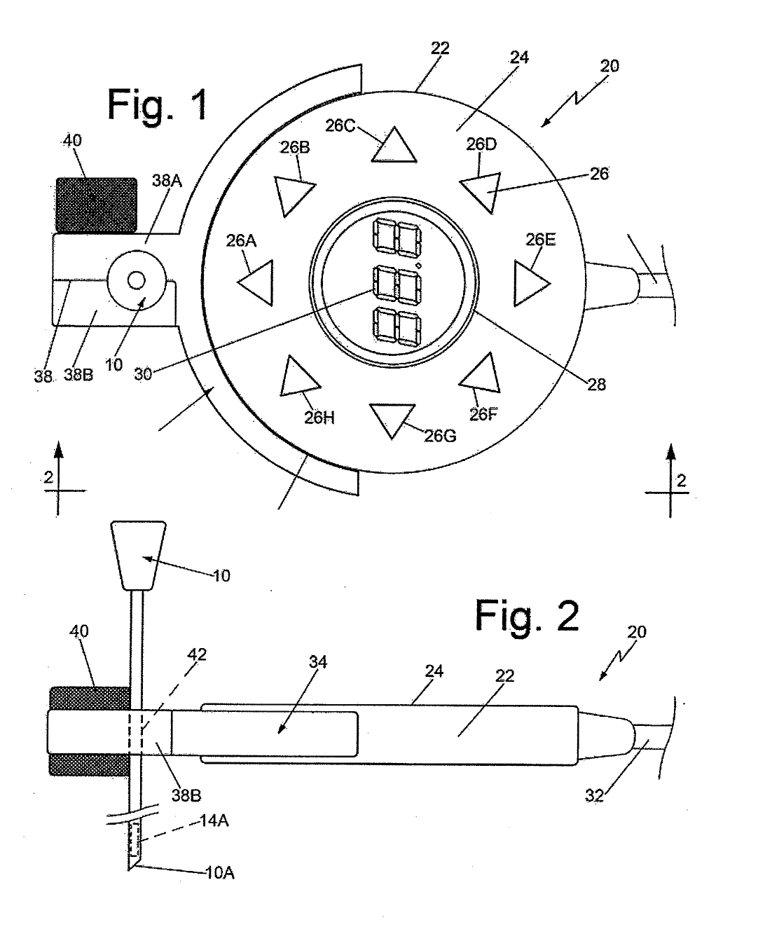

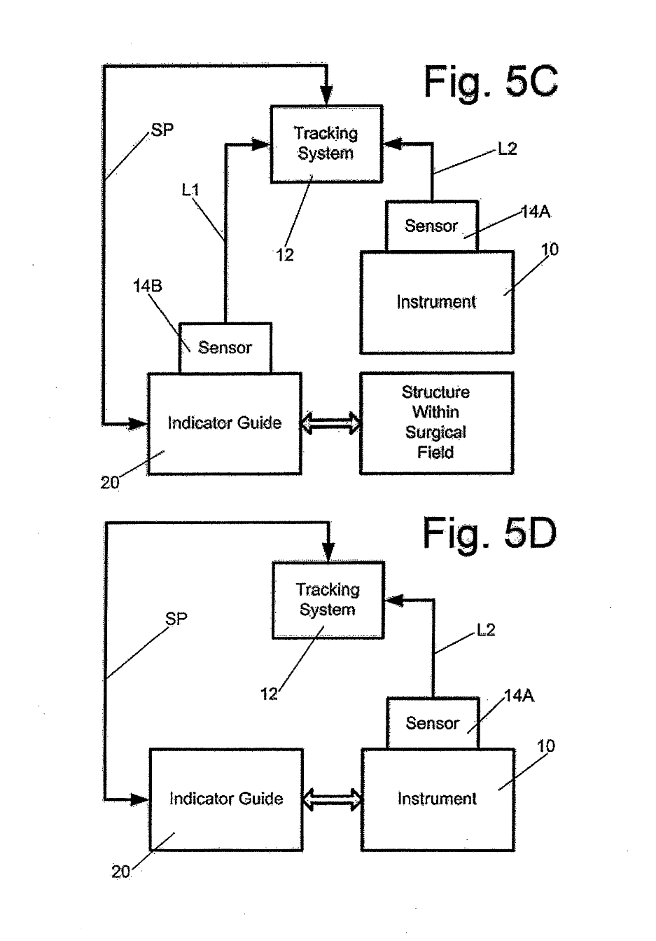

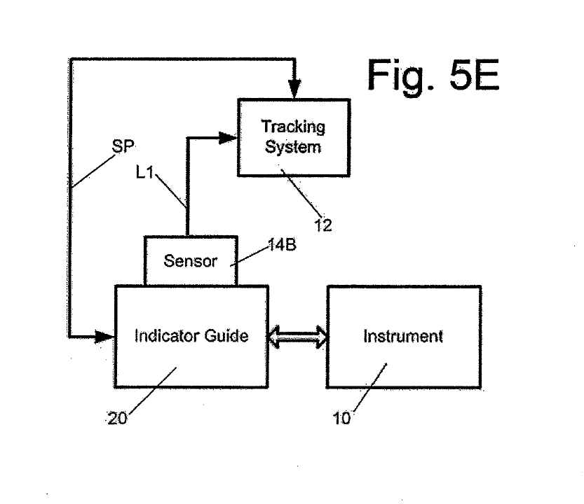

Referring now to the various figures of the drawing wherein like reference characters refer to like parts, there is shown at 20 in FIG. 1 one exemplary embodiment of an indicator guide for use in conjunction with an instrument 10 used in an image guided diagnostic or therapeutic procedure. The indicator guide 20 is arranged to be releasably secured to the instrument 10, e.g., a biopsy needle, and coupled to an electronic (e.g., computerized) tracking system 12 for accomplishing the image guided procedure on a patient as shown by the block diagram of FIG. 5A. In that diagram the releasable securement of the indicator guide 20 to the instrument 10 is represented by the double headed arrow. In practice that releasable securement can be accomplished in various ways. For example, the releasable securement can be accomplished by means of an adaptor 34 (to be described later) and which is shown in FIG. 1. Alternatively, it can be accomplished by means of an adhesive tape strip 44 (also to be described later) shown in FIG. 4. In FIG. 5B the indicator guide is shown arranged for releasable securement to the user's hand holding the instrument, e.g., the back of the user's hand, his/her wrist, etc. In FIG. 5C the indicator guide is shown arranged for releasable securement to some structure within the surgical field, e.g., to a surgical drape. Irrespective of how and to what the indicator guide is secured, when so secured it provides direct intuitive, visual indications to the user of the instrument on how to direct the instrument to the target within the patient's body and without the user having to remove his/her eyes from the surgical field or the instrument. In particular, the indicator guide provides visual pointers to the user as to the orientation and direction that the user should manipulate the instrument to in order to have a direct path to the target. The tracking system calculates that path and provides output signals representative thereof via a signal path SP (which may be a cable or wirelessly) to the indicator guide for the indicator guide to display.

The tracking system 12 can be of any suitable construction, such as those commercially available today. In the exemplary embodiment shown in FIG. 5A the tracking system includes a pair of EM sensors 14A and 14B, one of which is coupled to the instrument and one of which is coupled to the indicator guide. In particular, as seen in FIG. 1, the EM sensor 14A is located within the tip 10A of the instrument 10 and provides signals representing its position and orientation via line L2 to the tracking system 12. The sensor 14A may be located at some other position with respect to the instrument so long as it is coupled to the instrument in such a manner as to be movable with the instrument and its distance from the instrument's tip 10A is known and part of the data in the tracking system. In the exemplary embodiment of the indicator guide shown in FIG. 1, the EM sensor 14B is located within the housing making up the indicator guide, but it could be located outside of the indicator guide, so long as it is coupled to the indicator guide for movement with the indicator guide. The sensor 14B provides signals representative of its position and orientation via line L1 to the tracking system 12.

It should be noted at this juncture that the sensors 14A and 14B can be located at different positions with respect to the instrument and indicator guide, respectively, so long as their respective positions with respect to those components are known by software making up the computerized tracking system 12.

The use of the indicator guide 20 of this invention can be achieved in various ways, as will be described later. Suffice it for now to state that each method requires a 6DOF EM sensor affixed to the indicator guide and/or a 6 or 5DOF sensor affixed to the instrument, e.g., located in the tip of the instrument/needle. Either arrangement may be sufficient depending upon the instrument/needle being used and with the correct software or guide setup. However, it is preferred that two such sensors be used for reasons to be described later.

The methods of use of the indicator guide of this invention also requires a target that is well defined in the images and which can be marked and recognized in the 3-D data set of the tracking system software. Ideally, the target should remain in view of the tracked imaging transducer in the case of real-time ultrasound guidance, or the target needs to be recognized and clearly defined using image recognition software working in the digital image data set of the tracking system software. In either case, the target must be clearly located in the 3-D image volume for the already registered indicator guide to function as described. This is no different from what already exists in the practice today. In this regard, target visualization and marking is already required for all current EM image guided targeting software programs. The paradigm shift for the operator (user) provided by the present invention is the transfer and translation of navigational information onto the instrument itself (or at least onto the surgical field, e.g., on the back of the user's hand), onto a drape or other structure in the surgical field, etc).

In order for the indicator guide 20 to work as designed, it must be in a known spatial relationship to the instrument, e.g., the tip 10A of the instrument, which will be guided by it. This relationship may be tracked in at least three different ways. For example, if as shown in FIGS. 5A, 5B, and 5C, there is a 5 or 6DOF EM sensor in instrument tip (or attached at some other known position with respect to the instrument) and there is a 6DOF EM sensor affixed to or part of the indicator guide, then the associated software of the tracking system can manage their relationship using the EM field. If there is a 6 DOF EM sensor in the instrument tip only, such as shown in FIG. 5D, and the electronic guide 20 is affixed to the instrument at a known distance from and with a fixed orientation to the instrument tip sensor the software can manage the relationship and provide the desired guidance. If there is a 6DOF EM sensor in the indicator guide only and not in the instrument, such as shown in FIG. 5E, and the indicator guide is affixed to the instrument in a known and constant spatial relationship to the instrument tip (e.g., the instrument cannot bend) the software can manage the relationship and provide the desired guidance.

It is currently envisioned that the optimum clinical setup using the electronic indicator guide of this invention is to make use of an instrument that has a 5DOF or 6DOF EM sensor 14A in its tip and a 6DOF EM sensor 14B in the indicator guide 20, like shown in FIG. 5A. This allows for the indicator guide to be clipped or otherwise secured to the instrument or, in fact, placed anywhere in the EM field and also in the operator's (user's) visual field that is most convenient (e.g., on the back of the operating hand or adjacent to the instrument entry point on the patient). A more high-tech solution is also envisioned. That solution entails visually projecting the data provided by the electronic guide onto the operative field using any suitable means, e.g., a laser.

As should be appreciated by those skilled in the art in the arrangements shown in FIGS. 5A, 5B, 5D and 5E the indicator guide 20 is releasably secured to either the instrument 10 or the user's hand and thus will move with the instrument as the instrument is moved since the user's hand will be moving the instrument. In the case where the indicator guide is releasably secured to some structure (e.g., a surgical drape) within the surgical field, such as shown in FIG. 5C, the indicator guide will be stationary, while the instrument is moved. In any of those cases the tracking software can and will provide the necessary instructions to the indicator guide to provide the user with a visual indication of the path to the target without the user having to lose sight of the instrument in the surgical field.

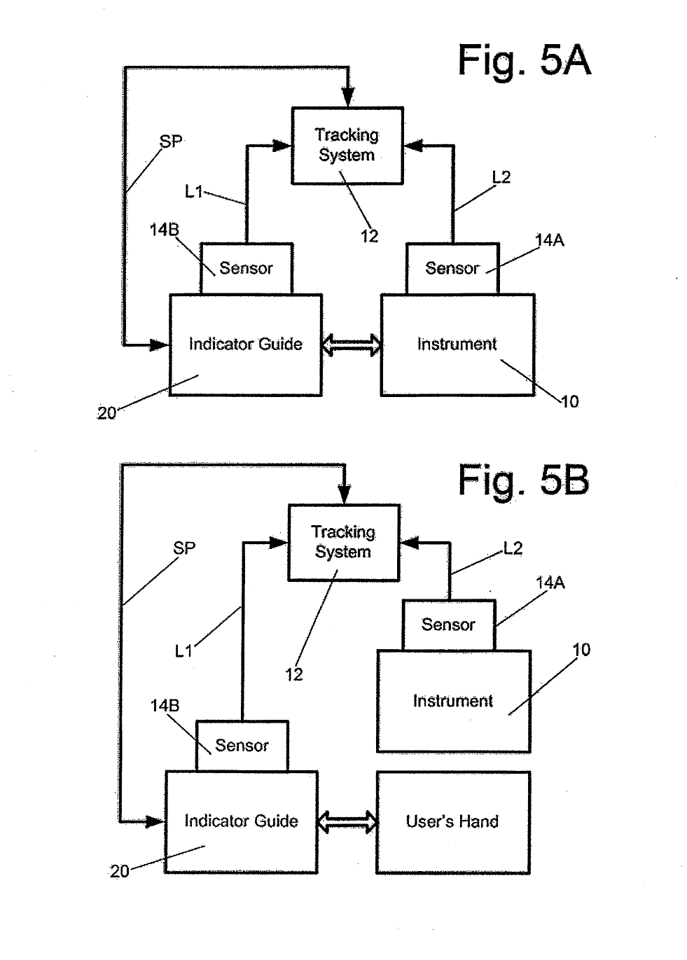

Turning now to FIGS. 1-4, the details of the exemplary embodiment of the indicator guide 20 will now be described. Thus, as can be seen therein the indicator guide basically comprises an electronic device housed in a body 22 having a front face in the form of a visual display 24. The display can take various forms, e.g., it may comprise a plurality of LEDs or liquid crystal display elements or may comprise an LED, liquid crystal or OLED screen providing various display elements. In the exemplary embodiment shown the display includes a directional display portion 26, an on-target display portion 28 and a distance display portion 30. The directional display portion 26 comprises a plurality, e.g., eight, directional display elements, e.g., LEDs, 26A, 26B, 26C, 26D, 26E, 26F, 26G and 26H. Those elements are arranged in a circular array so that they are equidistantly spaced from each other, with each display element pointing in a different respective direction. Each direction represents a potential path that the instrument 10 can be directed and moved along to bring the tip 10A of the instrument to the desired target, e.g., a lesion, within the body of the patient. To that end, each element is arranged to be illuminated to provide a visually perceptible signal to the user as to the direction he/she should move the instrument to bring the instrument to the target.

The on-target display portion 28 can take many forms and may be visual or audible or both. In the exemplary embodiment the on-target display portion 28 is visual to provide a visually perceptible signal to the user when the instrument is in a direct path and orientation to reach the target. In particular, it comprises a circular LED element that is located within the direction display array 26 and which illuminates to provide a visual signal to the user when the instrument is directly oriented in a path towards the target. As mentioned earlier, the on-target display can be audible. Thus, instead of providing a visual signal when the instrument is in the direct path to the target, the on-target display may provide an audible signal, e.g., a beep or series of beeps, etc., indicate that fact. An audible on-target signal may be provided in conjunction with a visual on-target signal.

In order to provide the user with information regarding the distance of the instrument, e.g., its tip 10A, to the target, the indicator guide includes the heretofore identified distance display 30 portion. In the exemplary embodiment the distance display portion 30 is in the form of a segmented digital numeric display (e.g., LED or liquid crystal) that is located within the center of the on-target display portion and provides a numeric readout of the distance of the instrument's tip 10A to the target. Like the on-target display 28, the distance display 30 may be in the form of an audible signal, e.g., a synthesized voice annunciating the distance to the target. That audible signal may be used in conjunction with the numeric visual display.

The electrical power and control signals to and from the electronic components making up the indicator guide 20 and the sensor 14A are provided via a strain relief-reinforced cable 32 extending out of the indicator guide's housing. The indicator guide 20 is connected to the tracking system 12 via the cable 32. It is also contemplated that the indicator guide be self-powered, e.g., battery powered, and wirelessly coupled to the tracking system.

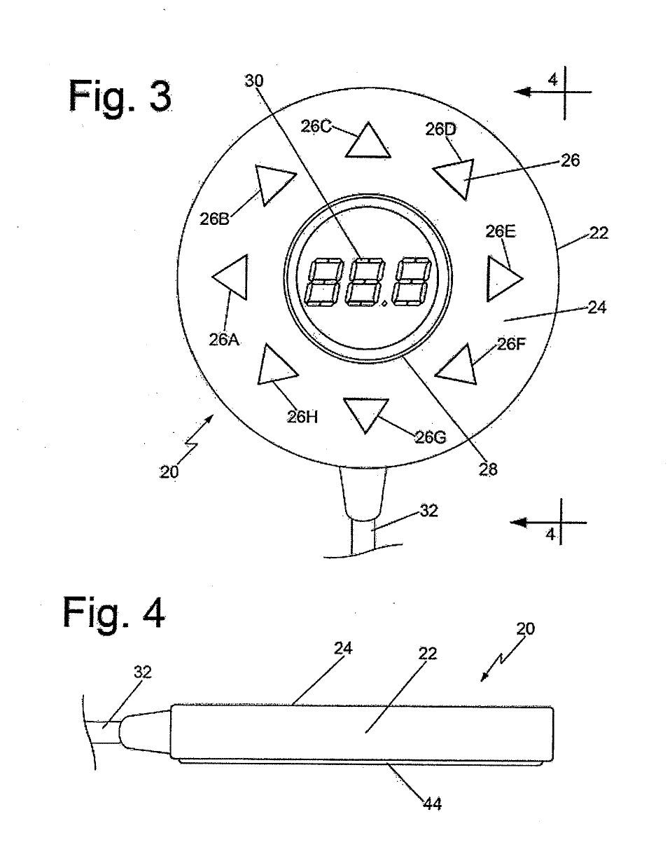

Turning now to FIGS. 1 and 2, one exemplary embodiment of an adaptor 34 for releasably securing the indicator guide 20 to the instrument 10 will now be described. Thus, as can be seen the adaptor 34 comprises an arcuate body having a recess 36 shaped corresponding to a portion of the outer periphery of the housing of the indicator guide. The arcuate body is releasably secured to the outer periphery of the indicator guide by any suitable means, e.g., a releasably securable adhesive, by cooperating VELCRO® hook and loop components or by any other suitable releasably securable means (not shown). The adaptor includes a clamp 38 having a pair of jaws 38A and 38B. The jaw 38A is a fixed extension of the arcuate body 34, while the jaw 38B is a movable member. A threaded rod (not shown) having a knurled knob 40 extends through the jaws 36A and 36B and is arranged when twisted to bring the movable jaw towards and away from the fixed jaw 36A, depending upon the direction that the knob is rotated. The interface of the two jaws is in the form of a pair of conjoining circular recesses, which form a passageway 42 (FIG. 2) through which the elongated body of the instrument (e.g., needle) 10 extends. Thus, when the needle 10 is extended through the passageway 42 and the knob 40 tightened, the indicator guide 20 will be fixedly secured to the instrument. The distance between the tip 10A of the instrument at which the sensor 14A is located and the position of the sensor 14B in the indicator guide 20 is provided to the tracking system 12 in order for the tracking system 12 to provide the required output signals to the indicator guide.

Turning now to FIG. 4 the details of the adhesive releasably securement means for releasably mounting the indicator guide to an instrument or to the operating hand of the user or to some fixed structure within the surgical field will now be described. To that end, as can be seen on the underside of the indicator guide (i.e., the side opposite the display 24), a strip of adhesive 44 is provided. If desired, the adhesive strip 44 may include a release liner (not shown) for protecting it until it is ready for use.

As should be appreciated by those skilled in the art from the foregoing the indicator guide 20, in effect, is a simple electronic pointer that visually shows the direction the instrument needle must be moved to reach the target, e.g., lesion, within the patient's body and also provides (e.g., shows) the distance of the tip of the instrument from the target. Thus, the indicator guide clearly indicates the required movement of the handled end of the instrument to direct the instrument tip into the target lesion and to stop at that point. In typical use, the guide 20 is used to get the instrument/needle pointed exactly at the target and then advance it. The guide, by being on or near the instrument/needle, allows the user to intuitively coordinate his/her hand movement with the electronic signal indicating direction because it is in the same direct field of view of the user. The guidance of the instrument becomes primarily directed by the indicator guide, not by some remotely located monitor (as has characterized the prior art). In particular, using the instrument guide 20 of this invention makes checking the “live” images on a remotely located monitor more elective and confirmatory, rather than mandatory and directive (as has characterized the prior art).

The methodology of this invention may make use of virtually the same software that currently is in common use today with conventional EM tracking systems. However, with the subject invention that software will be calculating the difficult spatial translations to lead the operator's movement of the instrument rather than the operator having to rely on his/her brain to reconstruct a mental 3-D image of the operative field to make those hand movements while watching the remote monitor to follow the moves suggested by the monitor's on-screen icons.

As with any image guided therapeutic or diagnostic procedure maintenance of a sterile operative field is necessary. Thus, to that end the use of the subject invention contemplates using a soft, clear plastic sterile cover (not shown) for the indicator guide, the associated EM sensor and the cabling. Alternatively, the core electronic components of the indicator guide and the 6DOF EM sensor could be potted together, have a single cable for re-use and have a sterile injection molded plastic housing that form the indicator dial face with an attached sleeve cover for the cable. It is also contemplated that the indicator guide can be made as a disposable device, although at this time such an arrangement is unlikely due to the inherent costs involved.

Without further elaboration the foregoing will so fully illustrate my invention that others may, by applying current or future knowledge, adopt the same for use under various conditions of service.

I claim:

1. An indicator guide for guiding the movement of a instrument by a user in an image guided medical procedure on a patient, the instrument being arranged to be introduced and guided in a surgical field to a target site within the body of the patient by an electronic tracking system, at least one of the instrument and said indicator guide comprising a sensor for providing a signal to the tracking system, the tracking system being arranged determine the position and orientation of the instrument with respect to the target site in response to the signal from the sensor and for providing output signals to said indicator guide, said indicator guide comprising a display responsive to the output signals from the tracking system for providing a visual indication of the path to which the instrument should be oriented and directed to reach the target site and for providing a perceptible indication of the distance of the instrument to the target site, said display being located on or immediately adjacent the instrument in the surgical field, whereupon the user can readily see the display while directly viewing and moving the instrument along that path.

2. The indicator guide of claim 1, wherein the tracking system comprises an EM tracking system and wherein the sensor is mounted on the instrument.

3. The indicator guide of claim 1 wherein the tracking system comprises an EM tracking system and wherein the sensor is mounted on said indicator guide.

4. The indicator guide of claim 1 wherein the tracking system comprises an EM tracking system and wherein a sensor is mounted on said indicator guide and a sensor is mounted on the instrument.

5. The indicator guide of claim 1 wherein said display comprises an array of direction indicators and an on-target indicator, said on-target indicator being responsive to said output signal from said tracking system to provide a perceptible indication to the user when the instrument is aimed in a direct path toward the target, said direction indicators comprising an array of plural visual indicators, each pointing in a respective, different direction representing a potential path along which the instrument may to be directed, said array being arranged so that the one of said plural second visual indicators indicating the path to the target is activated in response to said output signal to provide a visual indication to the user pointing in the direction of the path that the instrument is to be moved to the target.

6. The indicator guide of claim 5 wherein said on-target indicator is a visually perceptible indicator.

7. The indicator guide of claim 5 wherein said on-target indicator is an audibly perceptible indicator.

8. The indicator guide of claim 1 wherein said a perceptible indication of the distance of the instrument to the target site is directly visually perceptible by the user.

9. The indicator guide of claim 1 wherein said a perceptible indication of the distance of the instrument to the target site is audibly perceptible by the user.

10. The indicator guide of claim 1 wherein the perceptible indication of the distance of the instrument to the target site comprises visual indicia in said display.

11. The indicator guide of claim 1 wherein the perceptible indication of the distance of the instrument to the target site comprises an audible signal.

12. The indicator guide of claim 1 wherein said indicator guide is arranged to be releasably secured to the instrument.

13. The indicator guide of claim 1 wherein said indicator guide is arranged to be releasably secured to the hand of the user.

14. The indicator guide of claim 1 wherein said indicator guide is arranged to be releasably secured to a structure within the surgical field.

15. The indicator guide of claim 12 additionally comprising an adaptor for releasable securement of said indicator guide to the instrument.

16. The indicator guide of claim 12 additionally comprising an adhesive for releasable securement of said indicator guide to the instrument.

17. The indicator guide of claim 13 additionally comprising an adhesive for releasable securement of said indicator guide to the hand of the user.

18. The indicator guide of claim 14 additionally comprising an adhesive for releasable securement of said indicator guide to the structure within the surgical field.

19. A method for guiding the movement of an instrument by a user in an image guided medical procedure on a patient, the instrument being arranged to be introduced and guided in a surgical field to a target site within the body of the patient by an electronic tracking system, the tracking system including a sensor and being arranged determine the position and orientation of the instrument with respect to the target site in response to a signal from the sensor and for providing output signals indicative thereof, said method comprising;
providing a instrument guide comprising a display, said display being responsive to the output signals from the tracking system for providing a visual indication of the path to which the instrument should be oriented and directed to reach the target site and for providing a perceptible indication of the distance of the instrument to the target site;
coupling the sensor to at least one of the instrument and said indicator guide; and
disposing said display on or adjacent said instrument in the surgical field to be directly visible within the surgical field with said instrument, whereupon the user can readily see the display while directly viewing and moving the instrument along that path.

20. The method of claim 19 wherein the tracking system comprises an EM tracking system and wherein the sensor is mounted on the instrument.

21. The method of claim 19 wherein the tracking system comprises an EM tracking system and wherein the sensor is mounted on the indicator guide.

22. The method of claim 19 wherein the tracking system comprises an EM tracking system and wherein a sensor is mounted on the indicator guide and a sensor is mounted on the instrument.

23. The method of claim 19 wherein said display comprises an array of direction indicators and an on-target indicator, said on-target indicator being responsive to said output signal from said tracking system to provide a perceptible indication to the user when the instrument is aimed in a direct path toward the target, said direction indicators comprising an array of plural visual indicators, each pointing in a respective, different direction representing a potential path along which the instrument may to be directed, said array being arranged so that the one of said plural second visual indicators indicating the path to the target is activated in response to said output signal to provide a visual indication to the user pointing in the direction of the path that the instrument is to be moved to the target.

24. The method of claim 23 wherein said on-target indicator is a visually perceptible indicator.

25. The method of claim 23 wherein said on-target indicator is an audibly perceptible indicator.

26. The method of claim 19 wherein said a perceptible indication of the distance of the instrument to the target site is visually perceptible by the user.

27. The method of claim 19 wherein said a perceptible indication of the distance of the instrument to the target site is audibly perceptible by the user.

28. The method of claim 26 wherein the distance to the target is displayed by numeric indicia in said display.

29. The method of claim 19 wherein said indicator guide is releasably secured to the instrument.

30. The method of claim 19 wherein said indicator guide is releasably secured to the hand of the user.

31. The method of claim 19 wherein said indicator guide is releasably secured to a structure within the surgical field.
